(19) United States
(12) Patent Application Publication
(10) Pub. No.: US 2014/0107473 A1
(43) Pub. Date: Apr. 17, 2014

(54) Laser Guidance System for Interventions

(71) Applicant: Cincinnati Children's Hospital Medical Center, Cincinnati, OH (US)

(72) Inventors: Charles L. Dumoulin, Cincinnati, OH (US); Neil Johnson, Cincinnati, OH (US); Ronald Pratt, Erlanger, KY (US)

(73) Assignee: Cincinnati Children's Hospital Medical Center, Cincinnati, OH (US)

(21) Appl. No.: 14/055,254

(22) Filed: Oct. 16, 2013

Related U.S. Application Data
(60) Provisional application No. 61/714,815, filed on Oct. 17, 2012.

(51) Int. Cl. A61B 19/00; A61B 17/17; A61B 17/00; A61B 6/12; A61B 8/08

(52) U.S. Cl. CPC A61B 19/201; A61B 6/12; A61B 19/5212; A61B 8/0841; A61B 17/00234; A61B 17/1703
USPC 600/424; 606/130

(57) ABSTRACT

An instrument guide apparatus for use with a surgical or other guided intervention of a patient utilizing a guided instrument is disclosed. The instrument guide apparatus includes at least two line lasers mounted above the patient and generating intersecting planar laser lines along an instrument axis of entry in three-dimensional space above the patient. In an embodiment, the instrument axis of entry in three dimensional space above the patient is received from an imaging apparatus imaging the patient's body at least along a portion of the instrument's axis of entry into the patient. In an embodiment, the instrument axis of entry in three-dimensional space above the patient is a line-of-site for a camera mounted above the patient and controlled by the imaging apparatus.

### FIELD OF THE INVENTION

The present disclosure relates to an apparatus or system to assist interventions in a catheterization laboratory setting. The apparatus guides a needle, or some other type of surgical instrument, along a path in three-dimensional space above a patient. The current disclosure may be used in conjunction with an imaging modality. The invention further relates to a method for performing interventions using the disclosed apparatus.

### BACKGROUND

Inserting a biopsy needle, screw, or other needle-shaped instrument into a patient involves complex direction of accurate placement and angle insertion. Traditionally, angiography methods have been used to assist in needle placement, which includes the use of a radio-opaque contrast agent within a body to be seen by imaging methods. The disclosed system assists in such interventions (e.g. advancement of a biopsy needle) with or without angiography.

The disclosed intervention assistance system may be used in conjunction with an imaging modality such as the system disclosed in US Publication 2012/0008741, “System and Method for Generating Images of a Patient's Interior and Exterior,” herein referred to as the “optical/x-ray machine.” The US Publication 2012/0008741 is incorporated herein by reference.

The optical/x-ray machine is a system for generating an image including information of both an interior and an exterior of a patient. The system includes an X-ray device for providing an X-ray image of a patient's interior, and may also include a camera responsive to a wavelength for providing a camera image of a patent's exterior. The camera may be supported by the X-ray device for establishing a determined spatial relationship between the camera and the X-ray device. The system may further include a spatial reference for spatially correlating the X-ray image and the camera image, where the spatial reference is detectable in the X-ray image and in the camera image. A data processor is configured for rendering the camera image and the X-ray image into a composite image on the basis of spatial reference. The processor may also use a software program and previously acquired X-rays images to display a line on a monitor to show the proposed trajectory.

The optical/x-ray machine may employ video cameras mounted on the X-ray system's solid state detector on one end of the system's C-arm, and displays on one or more monitors real-time video of an instrument being inserted into a patient superimposed over a previously taken X-ray image of the patient. During a procedure, 2D and 3D images of the patient may be taken and co-registered. At select times during an intervention, the X-ray imaging may be shut off, and one of the cameras may be activated. Because the location and orientation of the “line of sight” of each camera is known, the video image of each camera can be registered with the pre-acquired X-ray images, or other pre-acquired images. The C-arm may be repositioned after the X-ray image is obtained to bring the “line of sight” of a selected camera in alignment with the axis of the X-ray beam used to generate the pre-acquired X-ray images. The video image and the X-ray image may be merged on a display screen to provide a real-time visualization of the scene superimposed upon an X-ray reference that shows the internal features of the patient, e.g. bones.

The interventionist can then align a needle, trocar, drill, or other instrument visually while having the X-ray image as a reference. While the optical/x-ray machine allows an operator to view the instrument as it is being inserted in relation to the target (tissue, bone, tumor, etc.), it displays the instrument within the patient as it is moving along its trajectory. Thus, the video image provides feedback to where the instrument is currently located, and does not provide a guide for the instrument to be inserted. The operator controls the direction of the instrument using feedback from the monitor(s).

One challenge to this approach is that both the X-ray and video images are inherently 2D. Additional information about the three-dimensional alignment of the tool and its anticipated 3-D trajectory in the body are not readily apparent. To overcome this, a second display having a different camera perspective and X-ray image can be employed. However, this dual scene approach requires expertise and the ability to mentally integrate these different perspectives into a 3D whole, resulting in trial and error when performing interventions.

The camera guide system of the optical/x-ray machine uses one or more cameras and monitors to capture movement in multiple directions. The patient is X-rayed, and the monitors display the instrument as it is inserted or drilled in to help an interventionist navigate the instrument to the proper spot and position. The system activates the camera closest to the activity, or target zone. Placing the needle, drill, or trocar is sensitive work because movement of the needle properly as it appears on one screen may be the wrong direction on another screen. Thus, there is a steep learning curve for needle placement.

The current camera guide system uses software to incorporate a digital guide lines on each monitor. Each camera's monitor will display one digital guide line, and by moving the needle around a radiologist aims to match the trajectory area in three of the monitors.

### SUMMARY OF THE INVENTION

An instrument guide apparatus of the current disclosure may take advantage of the ability of the optical/x-ray machine to position a camera towards the patient along a desired insertion axis (in three-dimensional space) for the surgical device or instrument. The instrument guide apparatus of the current disclosure provides a visual guide for an interventionist to follow in real space (not on a monitor), and appears directly on the patient (or directly upon any object placed along the insertion path/axis above the patient). The apparatus of the current disclosure may be mounted adjacent to the camera of the optical/x-ray machine, so the apparatus and camera may be simultaneously adjusted. The optical/x-ray machine may provide feedback to verify the instrument is being properly inserted. In addition, through use of the guide apparatus of the current disclosure, a camera component may no longer be necessary with the optical/x-ray machine, since the mechanisms for positioning the cameras may be alternatively utilized to orient the instrument guide apparatus.

The instrument guide apparatus of the current disclosure may be used with any imaging or intervention system, or as an independent system. For example, the instrument guide apparatus of the current disclosure may be used with an ultrasound imaging or intervention system. An example of such an ultrasound imaging and intervention system is disclosed in US Pub. No. 2006/0184029, the disclosure of which is incorporated herein by reference.

Although the cameras and monitors offer visual feedback of the instrument's trajectory within the patient's interior, the cameras and/or monitors are not necessarily present with the current disclosure, as the line of sight information generated for positioning the camera may be used to orient the instrument guide apparatus of the current disclosure.

In an embodiment, the disclosed instrument guide apparatus may generally be used in conjunction with a system, such as the optical/x-ray machine, involving X-ray interventions that merges real-time video images with 2D and 3D X-ray images. It may be incorporated to an existing video and X-ray system such, as the optical/x-ray machine, as an enhancement for image guidance. Benefits of this apparatus and method include facilitating surgery through small holes. It may be used in orthopedics to drill holes in bones, so a patient may no longer need to be opened up for a major operation.

An imaging guide apparatus associated with a surgical or other guided intervention of a patient using a guided instrument according to the current disclosure may include; an imaging apparatus generating an instrument axis of entry in three-dimensional space above a patient; and a pair of line lasers mounted above the patient generating intersecting laser lines along the instrument axis of entry.

An instrument guide apparatus, associated with a surgical or other guided intervention of a patient using a guided instrument, according to the current disclosure, may include a pair of line lasers mounted above the patient generating intersecting laser lines along a camera line of sight axis above the patient.

A method, associated with the current disclosure, for guiding an instrument for surgery or some other patient intervention, may include the steps of: receiving information pertaining to an instrument axis of entry in three-dimensional space above the patient; and generating a pair of planar laser beams that intersect along the instrument line of entry.

An instrument guiding apparatus according to the current disclosure may include a first planar, or line-forming, laser, and a second planar, or line-forming, laser mounted on either side of a video camera mounted the end of a mount above the patient, such as a C-arm. The camera and lasers may be mounted on a frame and the lasers may be able to swivel on the frame. The lasers may be offset from the camera's center by a fixed amount and are oriented such that the intersection of the two planar beams generated by the respective lasers is along the video camera's line of sight. As a result, a cross image will be formed by the two laser beams on any object placed between the lasers and the patient along the camera's line of sight, thereby providing visual assistance to a practitioner to guide an instrument along the camera's line of sight to the patient so that the instrument enters the patient along the correct intervention axis.

The lasers may enhance the optical/x-ray system and software to provide visual assistance to practitioners for accurately inserting an instrument into a patient along a desired three-dimensional trajectory path above the patient. A pair of lasers according to the disclosure may be employed with each camera that may have a line of sight along the desired trajectory path. Existing software may choose which camera and laser unit to use for the insertion line of sight. The camera images may be superimposed over previously taken X-ray images for reference.

An instrument guide apparatus for use with a surgical or other guided intervention of a patient utilizing a guided instrument is disclosed. The instrument guide apparatus includes at least two line lasers mounted above the patient and generating intersecting planar laser lines along an instrument axis of entry in three-dimensional space above the patient. In an embodiment, the instrument axis of entry in three dimensional space above the patient is received from an imaging apparatus imaging the patient's body at least along a portion of the instrument's axis of entry into the patient. In an embodiment, the instrument axis of entry in three-dimensional space above the patient is a line-of-site for a camera mounted above the patient and controlled by the imaging apparatus. In an embodiment, the line lasers are mounted along respective radians extending from the instrument axis of entry (e.g., from the camera's line of sight if a camera is present), where the radians meet at the instrument axis of entry at an angle of less than 180°, and the line lasers are oriented such that their respective laser planes extend through and intersect at the instrument axis of entry. In an embodiment, the radians meet at an angle of 90°.

A method for guiding an instrument for surgery or some other patient procedure, according to the current disclosure, may include a step of generating a pair of planar laser beams that intersect along an instrument axis of entry in three dimensional space above the patient. In a more detailed embodiment, prior to the generating step, the method may further include a step of positioning at least two line lasers (for generating the pair of planar laser beams) along respective radians extending from the instrument axis of entry, where the radians meet at the instrument axis of entry at an angle less than 180°. In a further detailed embodiment, the method may further include a step of adjusting the line lasers so that the pair of planes of the planar laser beams are parallel with the instrument axis of entry. In a further detailed embodiment, the method may further include a step of rotating the line lasers so that the pair of planes of the planar laser beams intersect along the instrument axis of entry. Alternatively, or in addition, the radians may meet at the instrument axis of entry at a 90° angle. Alternatively, or in addition, the line lasers are coupled to a mount positioned above the patient; and the method further includes steps of: generating information pertaining to the instrument axis of entry by an imaging system imaging an internal structure of the patient along; and orienting the mount until the line lasers are positioned such that the pair of planar laser beams will intersect along the instrument axis of entry. In a more detailed embodiment, the information pertaining to the instrument axis of entry may correspond to line-of-sight information for a camera. Alternatively, or in addition, the method may further include a step of manually guiding the instrument to the patient along a line formed in three dimensional space above the patient by the two intersecting planar laser beams; and this step may be performed while viewing the internal structure of the patient using the imaging apparatus.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only certain embodiments in accordance with the disclosure and are, therefore, not to be considered limiting of its scope, the disclosure will be described with additional specificity and detail through use of the accompanying drawings.

In the drawings:

FIG. 1 shows an exemplary instrument guide apparatus mounted on an imaging modality;

FIG. 2 shows an exemplary instrument guide system as it may be used with an example target;

FIG. 3 shows an exemplary instrument guide apparatus and system in connection with an imaging system, including a camera display monitor;

FIG. 4 shows an example instrument being inserted into a tissue from a camera view;

FIG. 5 shows an example instrument being inserted into a tissue from a merged camera and X-ray view;

FIG. 6 shows an X-ray image;

FIG. 7 shows an example instrument being inserted into a tissue from both a camera view and a merged camera and X-ray view; and

FIG. 8 shows a block diagram representation of an exemplary positioning of lasers with respect to a camera line-of-site and/or instrument trajectory in an exemplary embodiment.

### DETAILED DESCRIPTION OF THE DRAWINGS

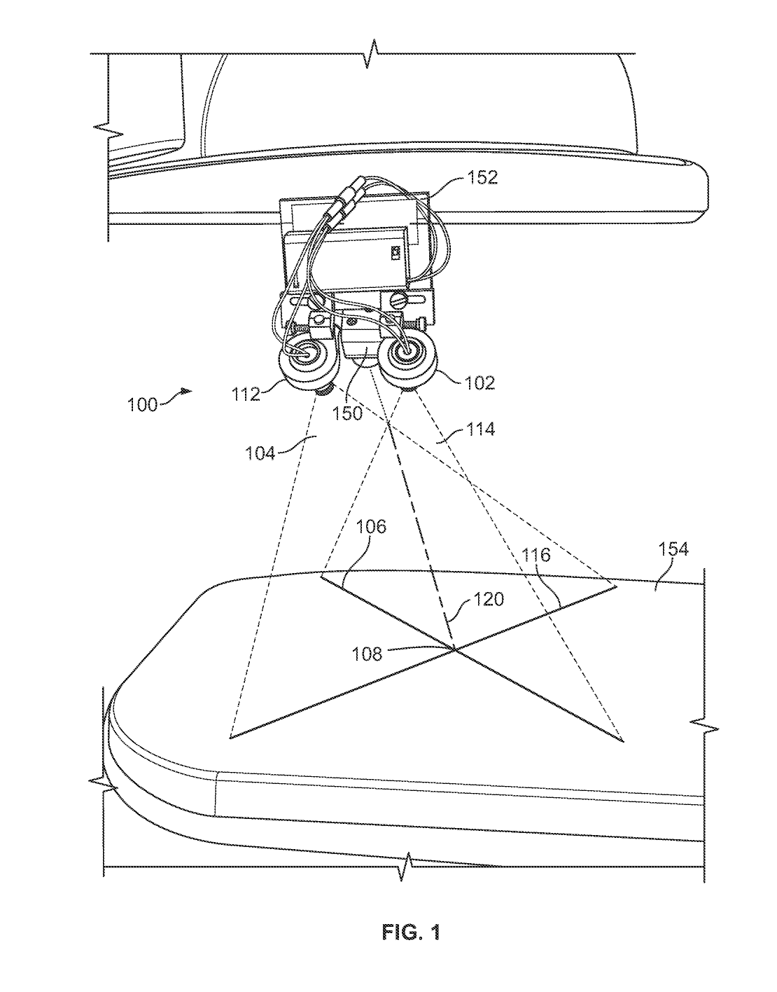

FIG. 1 shows an example instrument guiding apparatus 100 with line lasers 102 and 112 mounted on either side of camera 150 on supporting bracket, or mounting unit 152. Lasers 102 and 112 may be offset from camera 150 to allow for a clear line of vision for the camera. Lasers 102 and 112 output visible lines 106 and 116 on a surface, such as on table 154, or on a patient or on an object positioned between the lasers 102, 112 and the patient or table. The lasers 102, 112 are mounted and oriented so as to project intersecting planar laser beams 104, 114, where the intersection of the planar laser beams is along a line of sight of the camera and/or is along a line of trajectory in three dimensional space for a surgical instrument that is to be introduced into a patient along that same trajectory. In projecting a laser line to a surface, lasers 102 and 112 each produce a laser plane 104 and 114, which may not be visible until it reaches a surface to reflect light. Each laser plane 104 and 114 may cross the line of vision of camera 150 so the intersection of laser planes 104 and 114 would occur along the line of sight of the camera 150. This intersection of planes 104 and 114 would be visible as an intersection line 120 projected along the line of sight of the camera, which serves as visual guide. Any object positioned between the lasers 102, 112 and the patient would have projected upon it an “X” (or cross-hair 108) formed by the two lasers, were the intersection point of the lines of the “X” is positioned along the line of sight of the camera and/or along the three-dimensional introduction trajectory for the surgical instrument. As a result, the laser planes 104, 114 and intersection line 120 can provide visual assistance to a practitioner to guide an instrument along a trajectory line between the lasers 102, 112 and the patient.

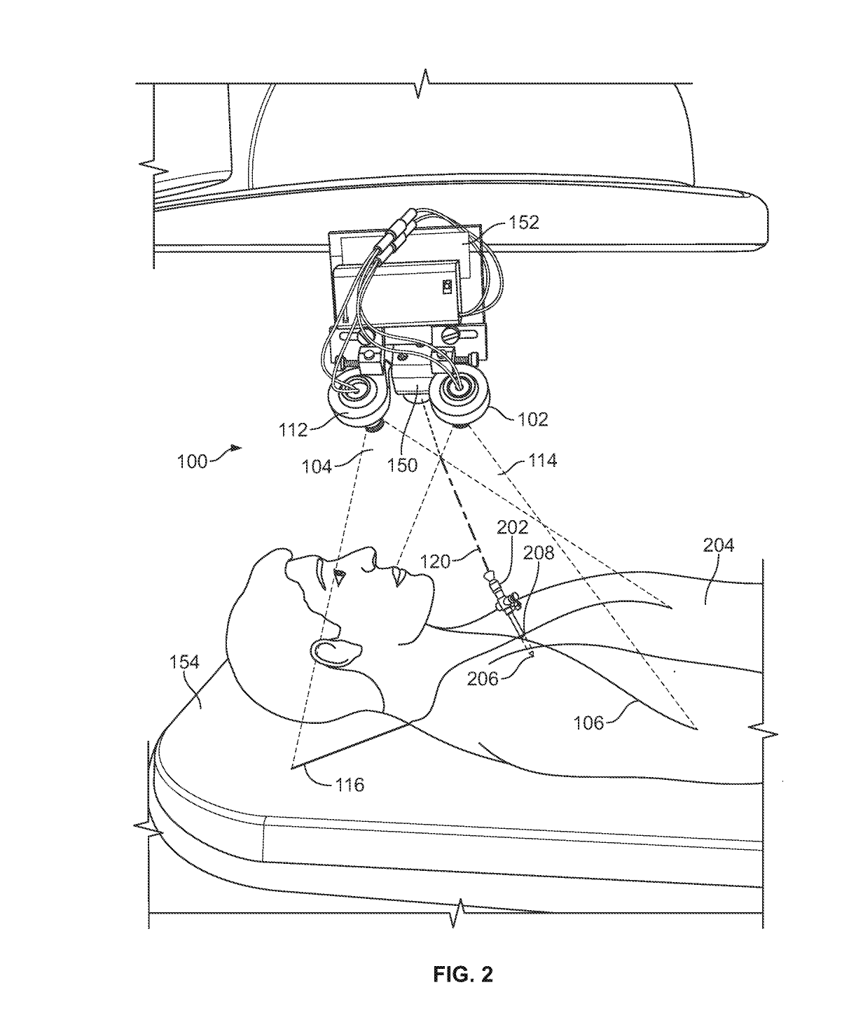

FIG. 2 shows an instrument guiding apparatus 100, including patient 204 on table 154. A target 206 is determined within patient 204, and mounting unit 152 is adjusted so camera 150 is pointed to target 206. As the first obstruction surface to laser planes 104 and 114, laser lines 106 and 116 are visible on patient 204. Intersection line 120 provides a visual guide to camera 150's line of vision so an instrument 202 may be navigated to target 206 at the proper position and entry angle. At the surface, intersection of lines 106 and 116 is a crosshair 208 marking the insertion, or entry point of instrument 202. When instrument 202 is aligned with guide 120, target 206 may be reached by instrument 202 with the proper angle and position.

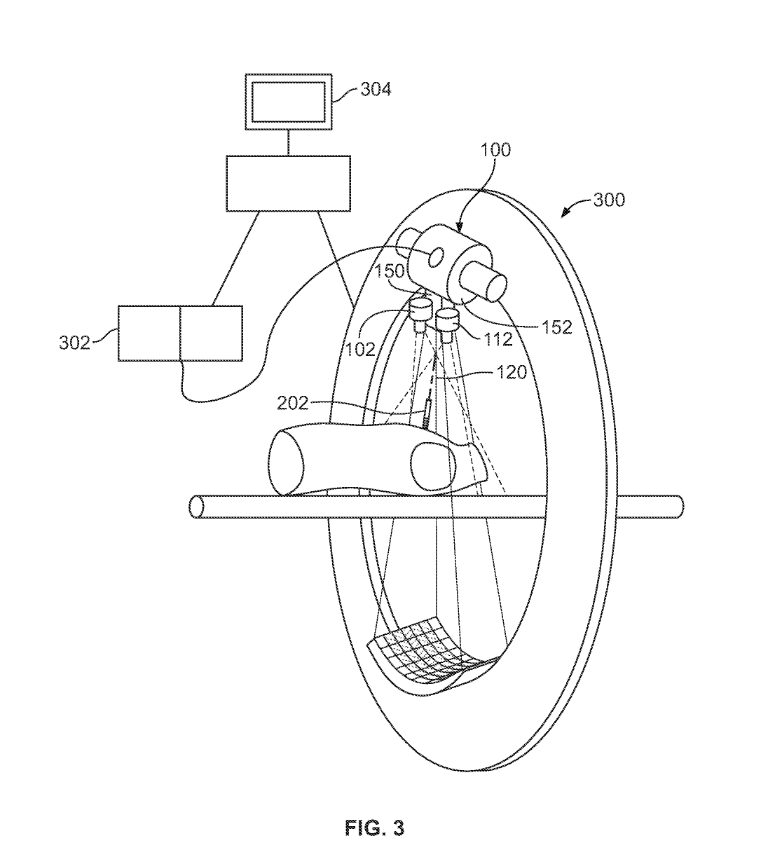

FIG. 3 depicts an exemplary imaging system 300 that instrument guiding apparatus 100 may form a part of Video from camera 150 may be transmitted to a processor 302, displayed on monitor 304, and superimposed over an acquired image. Imaging system 300 may be comprised of an X-ray, Computed Tomography, Magnetic Resonance, Ultrasound imaging system or the like. Referring to FIGS. 2 and 3, imaging system 300 may be configured to generate instrument trajectory information (e.g., pertaining to instrument insertion/introduction trajectory line above and into patient), and may then be configured to orient the mounting unit 152 above the patient so that the camera 150 line-of-sight is along the instrument trajectory line based upon that instrument trajectory information. Because the line lasers 102, 112 are positioned and oriented to emit planar laser beams 104, 114 that intersect along the camera's line-of-sight, the imaging system 300 may be used to guide the insertion/introduction of instrument 202 as described above.

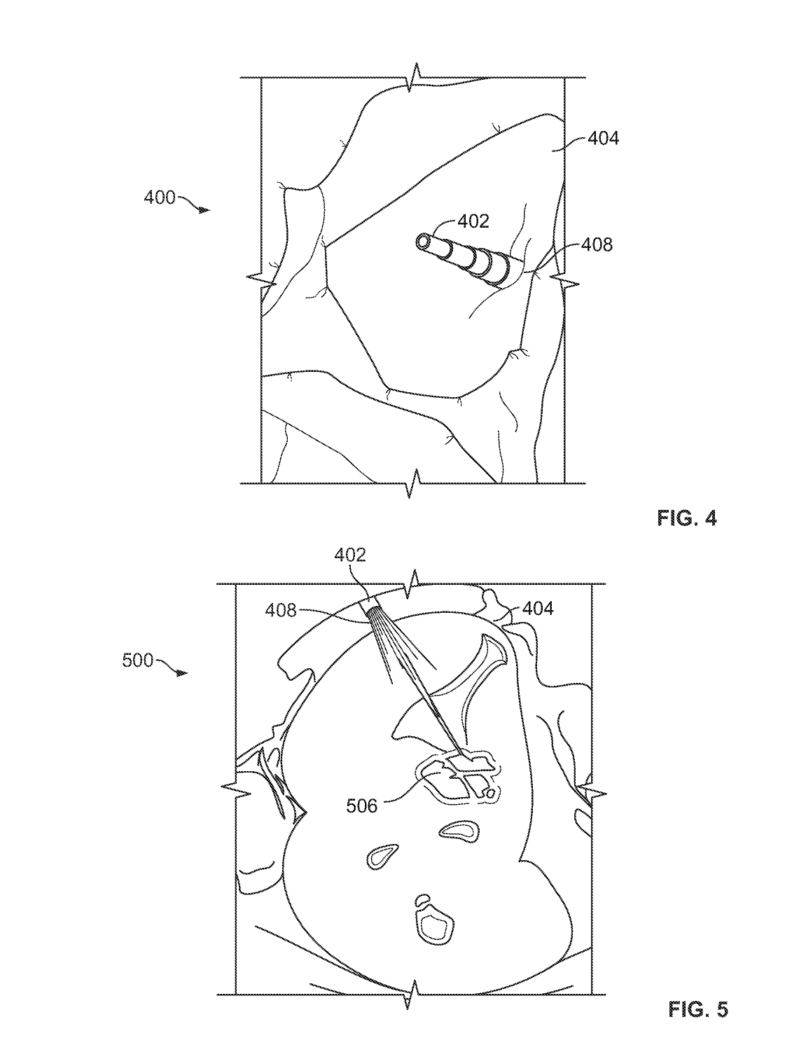

FIG. 4 displays an exterior view 400 of a patient 404 displayed on a monitor 304. Instrument 402 enters patient 404 at insertion point 408.

FIG. 5 shows a merged camera and X-ray view 500 of patient 404, including target 506. Instrument 408 can be seen within patient 404, having passed through insertion point 408.

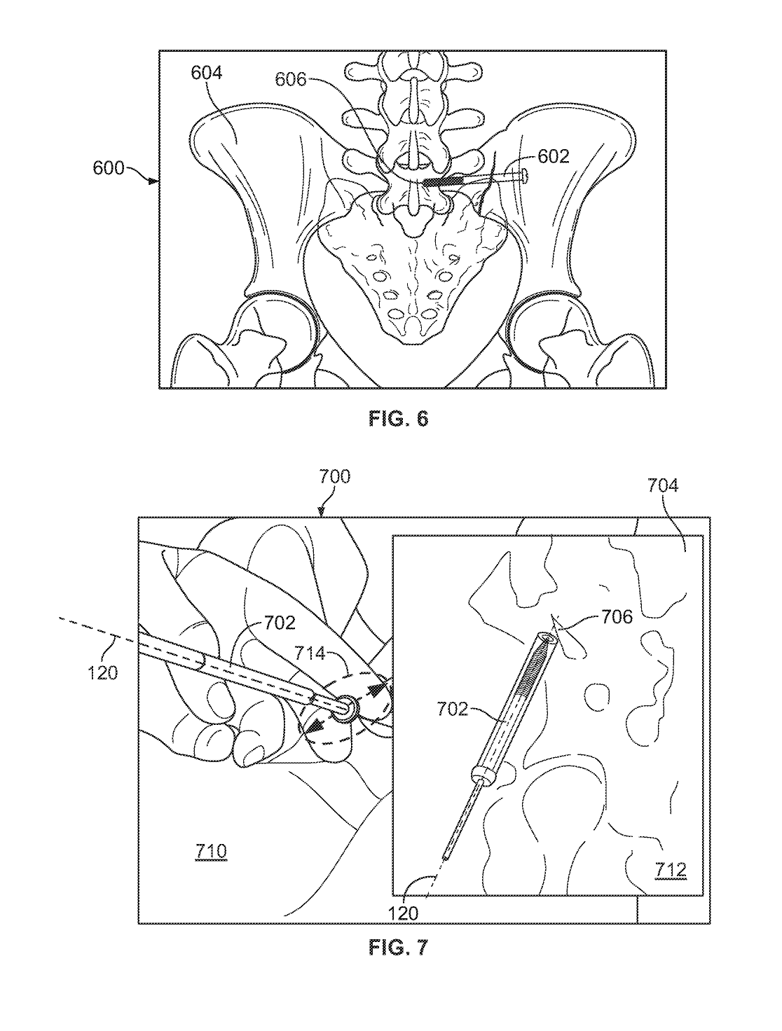

FIG. 6 shows an X-ray 600 of patient 604, including a screw 602 and a target point 606.

FIG. 7 shows composite view 700 comprised of a camera view 710 adjacent a merged camera and X-ray view 712. Instrument 702 is inserted into patient 704 towards target 706. It is appreciated that guide 120 and system 100 is useful because despite the merged view 712, the trajectory path of placement angle and position 714 is not properly guided by a 2D merged view 712. Line 120 marks the trajectory path in space, showing a technician the accurate trajectory and placement 714 of instrument 702.

In one embodiment, two lasers 102, 112 are each fitted with line-forming lenses so that each laser creates a plane 104, 114, visible as red lines 106, 116 when it hits a surface 154. The lasers 102, 112 are mounted on a frame 152 on either side of the camera 150, and offset from the camera in the same direction so as not to be in line with the camera 150. The axes of the lasers are adjusted to be parallel to the line-of-sight axis of the video camera. The lasers are rotated so that each laser line passes through the camera's axis (or line of sight). Although the two lasers 102, 112 are physically offset from the camera 150's center, the intersection of the laser lines occurs at the camera 150's line of sight axis. The intersection of the lasers defines the camera axis at all visual depths.

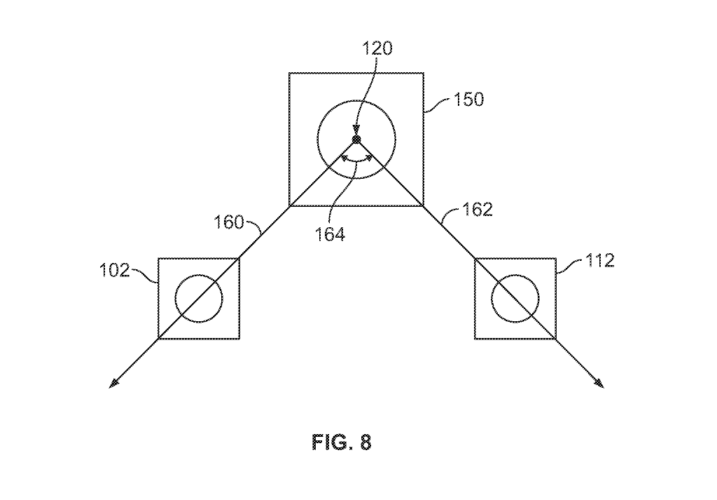

As shown in FIG. 8, an example placement of the lasers 102, 112 with respect to the camera's 150 center (line of sight) and/or with respect to the desired trajectory of the instrument is illustrated. In an embodiment, the lasers 102, 112 are mounted along respective radians 160, 162 extending from the instrument axis of entry (e.g., from the camera's line of sight if a camera is present), where the radians meet at the instrument axis of entry at an angle 164 of less than 180° (i.e, the lasers are not in line with the camera), and the lasers 102, 112 are oriented such that their respective laser planes extend through and intersect at the instrument axis of entry, which may be the camera line-of-sight, forming an intersection line 120. In an embodiment, the radians 160, 162 meet at an angle 164 of 90°.

Lasers of various manufacture and model may be used in the instrument guiding apparatus 100. The components used in a prototype of laser 102, 112 alignment fixture include two laser modules and lens from AixiZ OEM Electronics, specifically, AixiZ 650nm 12×30 mm laser module 3.2 VDC lasers, and AixiZ 120 Degree Line Lens for Standard 12×30 mm Laser Modules. Two nylon ball joint rod ends from McMaster-Carr are used in the laser alignment fixture.

The instrument guiding apparatus 100 of the disclosure overcomes the challenge of determining an instrument or needle position and angle placement by creating a planar laser intersection line 120 that is present at all locations along the length of each camera's “line of sight,” to assist radiologists in needle insertion into a tissue. Because the planar laser intersect line 120 is present along the entire visual depth of the camera, and shows up as cross hair 208 when it hits the surface of the patient (and the surface of any object positioned between the lasers and the patient, such as on the surgical instrument being guided), alignment of a needle or other surgical instrument 202 along the “line of sight” is straightforward, and may be performed with a single visual display. Aligning instrument 202 along the “line of sight” can be performed, for example, by placing the tip of the needle on the surface of the patient at the point where the cross hairs 208 appear, and then aligning the trailing end of the needle 202 until the trailing end is centered along the planar laser intersection line 120. In this configuration, the instrument 202 becomes aligned with the “line of sight” of the camera 150 and/or with the desired trajectory of the needle in three-dimensional space above the patient. Because imaging system 300 can determine where this desired trajectory line 120 (which may be the same trajectory line as the camera line-of-sight) is in relation to the pre-acquired X-rays 600, and because this line 120 can be used as a needle planning trajectory, the alignment line 120 and cross hair 208 provides a way to align instrument 202 using the pre-acquired X-ray images 600 without exposing the interventionist or the patient to additional X-rays.

This apparatus and method may reduce guesswork to guide an instrument such as a needle, pin or screw to the target because the desired path for the instrument 202 is along the intersection line 120 created by the intersection of the laser planes 104, 114. The laser intersection of the planes defines a line 120 in space. The laser lines 106, 116 may not be visible until it hits a surface 204. For example, to see the crosshair point 208 in space, a sterile tape may be used to bring the laser to visibility. The apparatus 100 provides a guide to align the needle at proper trajectory path. In actual positioning, and using the apparatus 100 of the disclosure, cameras 150 may not be necessary.

It is within the scope of the current disclosure that more than two lasers may be utilized to form a laser beam guide as disclosed. For example, it is within the scope of the current disclosure that more than two intersecting planar laser lines could be configured to intersect along the line-of-sight of the camera and/or along the axis of entry for the medical instrument. It is also within the scope of the current disclosure that the intersecting axis of the two or more line lasers may be offset from the axis of entry, while still remaining parallel to the axis of entry (the amount of offset may vary depending upon the application, but it is envisioned that an offset of several centimeters may be the maximum offset). It is also within the scope of the current disclosure that a plurality of line lasers as disclosed may be utilized to draw a figure to circumscribe the axis of entry (or run parallel to the axis of entry), such as a triangle, square, pentagon, etc.

Having described the detailed embodiments with reference to the attached figures, it will be apparent that the described embodiments are only exemplary in nature and that modifications may be made without departing from the scope of the invention(s) as claimed in the appending claims. Further, the invention(s) according to the current disclosure are defined by the appended claims and it is not intended that any specific elements or limitations are to be read into the plain meaning of the claims.

What is claimed is:

1. An instrument guide apparatus, for use with a surgical or other guided intervention of a patient utilizing a guided instrument, the instrument guide apparatus comprising:
at least two line lasers mounted above the patient and generating intersecting planar laser lines along an instrument axis of entry in three-dimensional space above the patient.

2. The instrument guide apparatus of claim 1, wherein the instrument axis of entry in three dimensional space above the patient is received from an imaging apparatus imaging the patient's body at least along a portion of the instrument's axis of entry into the patient.

3. The instrument guide apparatus of claim 2, wherein the instrument axis of entry in three-dimensional space above the patient is a line-of-site for a camera mounted above the patient and controlled by the imaging apparatus.

4. The instrument guide apparatus of claim 3, wherein:
the at least two line lasers are mounted along respective radians extending from the line-of-site for the camera, the radians meeting at the line-of-sight axis at an angle of less than 180°;
the line lasers are oriented such that their respective laser planes extend through and intersect at the line-of-sight axis.

5. The instrument guide apparatus of claim 1, wherein
the at least two line lasers are mounted along respective radians extending from the instrument axis of entry, the radians meeting at the instrument axis of entry at an angle of less than 180°;
the line lasers are oriented such that their respective laser planes extend through and intersect at the instrument axis of entry.

6. The instrument guide apparatus of claim 5, wherein the radians meet at the instrument axis of entry at a 90° angle.

7. A patient imaging and instrument guide apparatus, comprising:
an imaging system providing data pertaining to a straight trajectory line in a three-dimensional space above a patient for guiding a surgical or other medical instrument along the trajectory line; and
an instrument guide apparatus including at least two line lasers mounted approximate to the patient and generating intersecting planar laser lines along straight trajectory line.

8. The patient imaging and instrument guide apparatus of claim 7, wherein
the at least two line lasers are mounted along respective radians extending from the trajectory line, the radians meeting at the trajectory line at an angle less than 180°; and
the line lasers are oriented such that their respective laser planes extend through and intersect at the trajectory line.

9. The patient imaging and instrument guide apparatus of claim 8, wherein the radians meet at the trajectory line at a 90° angle.

10. A method for guiding an instrument for surgery or some other patient procedure, comprising a step of generating a pair of planar laser beams that intersect along an instrument axis of entry in three dimensional space above the patient.

11. The method of claim 10, further comprising, prior to the generating step, a step of positioning at least two line lasers for generating the pair of planar laser beams along respective radians extending from the instrument axis of entry, the radians meeting at the instrument axis of entry at an angle less than 180°.

12. The method of claim 11, further comprising a step of adjusting the at least two line lasers so that the pair of planes of the planar laser beams are parallel with the instrument axis of entry.

13. The method of claim 12, further comprising a step of rotating the at least two line lasers so that the pair of planes of the planar laser beams intersect along the instrument axis of entry.

14. The method of claim 11, wherein the radians meet at the instrument axis of entry at an angle of less than 180°.

15. The method of claim 11, wherein the at least two line lasers are coupled to a mount positioned above the patient.

16. The method of claim 15, further comprising steps of:
generating information pertaining to the instrument axis of entry by an imaging system imaging an internal structure of the patient along; and
orienting the mount until the at least two line lasers are positioned such that the pair of laser beams will intersect along the instrument axis of entry.

17. The method of claim 16, wherein the information pertaining to the instrument axis of entry corresponds to line-of-sight information for a camera.

18. The method of claim 16, further comprising a step of manually guiding the instrument to the patient along a line formed in three dimensional space above the patient by the two intersecting planar laser beams.

19. The method of claim 18, wherein the manual guiding step is performed while viewing the internal structure of the patient using the imaging apparatus.

20. A method for guiding an instrument for surgery or some other patient procedure, comprising a step of generating at least two intersecting laser lines that intersect along an instrument axis of entry in three dimensional space above the patient.

21. The method of claim 20, wherein the at least two intersecting laser lines are respectively generated by at least two line lasers mounted above the patient and offset from, and not collectively in-line with the instrument axis of entry.

22. The method of claim 20, further comprising steps of:
generating information pertaining to the instrument axis of entry by an imaging system imaging an internal structure of the patient along; and
orienting the interesting laser lines so that they intersect along the instrument axis of entry based upon the information pertaining to the instrument axis of entry generated by the imaging system.

23. An instrument guide apparatus, for use with a surgical or other guided intervention of a patient utilizing a guided instrument, the instrument guide apparatus comprising:
at least two line lasers mounted above the patient and generating intersecting planar laser lines, wherein the intersection of the planar laser lines runs parallel to an instrument axis of entry in three-dimensional space above the patient.

24. The instrument guide apparatus of claim 23, wherein the intersection of the planar laser lines runs coaxial with the instrument axis of entry in three-dimensional space above the patient.

25. The instrument guide apparatus of claim 23, wherein the planar laser lines form a portion of a shape circumscribing the instrument axis of entry in three-dimensional space above the patient.
